(19) United States
(12) Patent Application Publication
(10) Pub. No.: US 2014/0370652 A1
(43) Pub. Date: Dec. 18, 2014

(54) TETRACENE TETRACARBOXYLIC DIIMIDES AND THEIR PREPARATION

(71) Applicant: INSTITUTE OF CHEMISTRY, CHINESE ACADEMY OF SCIENCES, Beijing (CN)

(72) Inventors: Zhaohui Wang, Beijing (CN); Wan Yue, Beijing (CN)

(21) Appl. No.: 14/376,868

(22) PCT Filed: Feb. 10, 2012
(86) PCT No.: PCT/CN2012/000160

§ 371 Date: Aug. 6, 2014

(51) Int. Cl. H01L 51/00; H01L 51/42; H01L 51/05; H01L 51/50

(52) U.S. Cl. CPC H01L 51/0072; H01L 51/0007; H01L 51/50; H01L 51/42; H01L 51/05; H01L 51/5072
USPC 438/99; 546/66; 546/41; 546/31; 546/26

(57) ABSTRACT

A new family of tetracene tetracarboxylic diimides is provided. These ones can made by reacting a 9-stannafluorene with a tetrabromo compound including a tetracene tetracarboxylic diimide core. They can be used as n-type electron-transporting materials in electronic devices such as n-channel field-effect transistors. They exhibit excellent air-stability and do not cause parasite injections of holes.

The present invention relates to compounds including a tetracene tetracarboxylic diimide moeity and to a method for preparing them. The present invention relates also to the use of these compounds as electron transporting material in devices such as organic field-effect transistors and organic electroluminescent devices. Finally, the present invention relates to various devices comprising the above compounds.

Tetracene has a large conjugated system, which was found to possess good transport properties of charges. Even at low temperature, the mobility of single crystal is about 0.02 cm²/Vs (Appl. Phys. Lett. 2004, 84, 5383-5385). On a substrate modified by octadecylchlorosilane, tetracene films having a mobility of about 0.1 cm²/Vs were prepared (Appl. Phys. Lett. 2002, 80, 2925-2927).

One known approach to improve the performances of acenes consists in integrating functional groups into the parent molecules. Thus, for example, the substitution of all the hydrogen atoms of pentacene with electrophilic fluorine atoms was shown to improve the stability of pentacene; in addition, the so-obtained perfluoropentacene exhibited n-type organic semiconductor properties (J. Am. Chem. Soc. 2004, 126, 8138-8140). However, the systematic change of effective functional groups on acenes, including tetracene and pentacene, poses a great challenge since there are very few synthetic methodologies that can serve this purpose, and these ones are further difficult-to-implement and expensive.

Certain modified perylenes and naphthalenes, which combine an aromatic core with strong electrophilic tetracarboxylic diimide substituents, were also reported for their promising n-type field-effect properties (Angew. Chem. Int. Ed. 2010, 49, 740-743; Nature, 2000, 404, 478-481).

Many materials of the prior art which could have otherwise been useful as “n-type materials”, including perylene tetracarboxylic diimides and naphthalene tetracarboxylic diimides, suffer however from a poor air-stability. The requirement in terms of air-stability for electron-transporting (“n-type”) materials is so important in practice that not complying with this requirement just makes the materials unsuitable for many OLED and OPV applications.

Qun Ye et alii (Organic Letters, 2011, vol. 13, No. 22, pages 5960-5963) prepared a tetrathienyl-fused tetracene tetracarboxylic diimide (TT-TDI) exhibiting a LUMO energy level of −4.10 eV. Based on the Applicant's own experience, compounds having a LUMO lower than −4.00 eV, such as Qun Ye's TT-TDI, usually exhibit suitable air-stability for processing and device operation in ambient conditions. Thus, in the light of its LUMO value, TT-TDI is deemed to exhibit suitable air-stability. Unfortunately, as reported by Qun Ye himself in the last paragraph of page 5962, devices based on TT-TDI exhibited n-type behavior under nitrogen only; on the other hand, in air—i.e. under practically important conditions—, the same TT-DDI based devices exhibited ambipolar character, with undesirable reduction of the charge carrier mobility and On/Off ratio. It is suspected that the hole transport was generated in ambient due to doping by molecular oxygen. It is then concluded in the Organic Letters reference, that the ambipolar charge transporting character must originate from the small bandgap of TT-DDI (E_{g}=1.52 eV).

As the skilled person can easily understand, tetracene tetracarboxylic diimides with a too low bandgap cannot work properly as “n-type materials” when exposed to ambient oxygen, as its effect of “natural doping” causes parasite transport of holes. To fix the ideas, based on the Applicant's experience, a bandgap of at least about 1.80 eV is in general necessary for an organic semiconductor to work properly exclusively as “n-type material”; on the other hand, n-channel field effect transistors that are prepared with compounds having a lower bandgap cannot switch with a clear cut from the “on” position, to the “off” one.

There remains thus still a strong need for improved electron transporting compounds. These ones should advantageously satisfy not only with the requirement of high air-stability but also the requirement of suitability for use in n-channel field-effect transistors and the like (exclusively electron-transporting materials, not causing parasite injection and transport of holes). Further, a simple and efficient process for the synthesis of the aforesaid compounds is in demand in the art.

To this end, the present invention concerns a tetracene tetracarboxylic diimide compound of formula I:

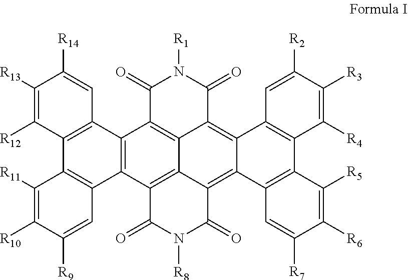

wherein:
R₁ and R₈, identical to or different from each other, are chosen from hydrogen, and C₁-C₃₀ organic groups, and
R₂, R₃, R₄, R₅, R₆, R₇, R₉, R₁₀, R₁₁, R₁₂, R₁₃ and R₁₄, identical to or different from each other and identical to or different from R₁ and R₈, are chosen from hydrogen, halogens and C₁-C₃₀ organic groups.

A single representative of the invented compound was disclosed by the Applicant prior to the filing date of the present application, while remaining totally silent on its particular merits, as will be explained in the “examples” section. The disclosure took place in the form of a 4-page communication of the Journal of the American Chemical Society entitled “One-pot synthesis of stable NIR tetracene diimides via double cross-coupling” which was published on web mid-October 2011 (Internet address: http://pubs.acs.org/JACS, reference “dx.doi.org/10.1021/ja207630a”) and in issue on Nov. 16, 2011 (J. Am. Chem. Soc., 2011, 133, 18054-18057). Authors thereof are from the Institute of Chemistry of Chinese Academy of Science have been designated as the inventors of the present invention. Supporting information, including additional experimental details, characterization data of compounds and measurements, can also be found at http://pubs.acs.org/JACS. The whole content of this communication and the related supporting information is hereby incorporated by reference for all purposes. The single representative of the invented compound which was disclosed by the Applicant in the aforesaid communication is compound 11, i.e. a compound of formula I wherein R₁ and R₈ are n-octyl and wherein R₂, R₃, R₄, R₅, R₆, R₇, R₉, R₁₀, R₁₁, R₁₂ and R₁₃ are hydrogen. Hence, in a particular embodiment, the present invention is directed to the tetracene tetracarboxylic diimide compound of formula I as above defined (“the invented compound” in general) with the exception of compound 11 of the aforesaid communication. The C₁-C₃₀ organic groups may be electron-withdrawing groups. The terms “electron-withdrawing groups” should be understood under their commonly accepted meaning, i.e. groups, which when incorporated, in the invented compound, are capable of withdrawing electrons. For the purpose of the present invention, halogen substituents, also named “halogenos”, are considered as groups, although containing a single atom.

The electron-withdrawing groups are advantageously chosen from cyano, C₁-C₃₀ acyls, halogens, C₁-C₃₀ perhalogenocarbyls, C₁-C₃₀ perhalogeno-oxycarbyls, C₁-C₃₀ partially halogenated hydrocarbyls having a halogen atom over hydrogen atom molar ratio of at least 0.50 and C₁-C₃₀ partially halogenated oxyhydrocarbyls having a halogen atom over hydrogen atom molar ratio of at least 0.50. C₁-C₃₀ perhalogenocarbyls include C₁-C₃₀ perhalogenoalkyls, C₆-C₃₀ perhalogenoaryls, C₆-C₃₀ perhalogenoalkylaryls and C₆-C₃₀ perhalogenoaralkyls; likewise, the previously cited C₁-C₃₀ perhalogeno-oxycarbyls, C₁-C₃₀ partially halogenated hydrocarbyls and C₁-C₃₀ partially halogenated oxyhydrocarbyls include halogenated alkyls, halogenated aryls, halogenated alkylaryls and halogenated aralkyls, with the possible presence of the —O— moiety and the appropriate degree of halogenation.

Except for halogens themselves, the number of carbon atoms of the electron-withdrawing groups used in accordance with the present invention may range from 1 to 20 carbon atoms, from 1 to 10 carbon atoms or from 1 to 5 carbon atoms. According to the invention, a preferred electron-withdrawing group is cyano; other preferred electron-withdrawing groups include fluorine, and are very preferably chosen from fluorine itself, C₁-C₁₅ perfluorocarbyls and C₁-C₁₅ partially fluorinated hydrocarbyls having a halogen atom over hydrogen atom molar ratio of at least 0.50; still more preferably, they are chosen from fluorine itself, C₁-C₈ perfluoroalkyls and C₁-C₈ partially fluorinated alkyls having a halogen atom over hydrogen atom molar ratio of at least 0.50; alkyls may be n-alkyls.

The C₁-C₃₀ organic groups may also be solubilizing groups. A solubilizing group can be defined as a group which, when present in the invented compound, increases the solubility of said invented compound in at least one solvent thereof, when compared to a reference compound, identical to the invented compound, except that the solubilizing group has been replaced by hydrogen, at a fixed temperature usually chosen in range from 15° C. to 50° C. Non limitative examples of solvents that may be used in accordance with the invention include monochlorobenzene, p-dichlorobenzene, dichloromethane, chloroform, toluene and tetrahydrofuran. Solubility tests to identify solubilizing groups may be notably achieved at room temperature (23° C.) or at 40° C. using p-dichlorobenzene as the reference solvent.

The solubilizing groups are advantageously chosen from C₁-C₃₀ hydrocarbyls, C₁-C₃₀ oxyhydrocarbyls, C₁-C₃₀ partially halogenated hydrocarbyls having a halogen atom over hydrogen molar ratio below 0.50 and C₁-C₃₀ partially halogenated oxyhydrocarbyls having a halogen atom over hydrogen molar ratio below 0.50. C₁-C₃₀ hydrocarbyls include C₁-C₃₀ alkyls, C₆-C₃₀ aryls, C₆-C₃₀ alkylaryls and C₆-C₃₀ aralkyls; likewise, the previously cited C₁-C₃₀ oxyhydrocarbyls, C₁-C₃₀ partially halogenated hydrocarbyl and C₁-C₃₀ partially halogenated oxyhydrocarbyls include halogenated alkyls, aryls, alkylaryls and aralkyls, with the possible presence of the oxy functionality and of halogen substituents under the appropriate degree of halogenation.

The number of carbon atoms of the solubilizing groups used in accordance with the present invention may range from 1 to 25 carbon atoms, from 1 to 15 carbon atoms or from 1 to 5 carbon atoms. According to the invention, preferred solubilizing groups are chosen from C₁-C₁₈ hydrocarbyls and C₁-C₁₈ partially fluorinated hydrocarbyls having a halogen atom over hydrogen atom molar ratio below 0.50; still more preferably, they are chosen from C₄-C₁₂ alkyls and C₄-C₁₂ partially fluorinated alkyls having a halogen atom over hydrogen atom molar ratio below 0.50; alkyls may be n-alkyls.

Preferably, at least one of R₁ and R₈ is a solubilizing group. More preferably, R₁ and R₈ are solubilizing groups. C₁-C₃₀ alkyl, especially C₄-C₁₂ alkyls, are good choices for R₁ and/or R₈ solubilizing group(s).

In the invented compound, R₄, R₅, R₁₁ and R₁₂ represent a preferably hydrogen atom (feature f1). It is also preferred that R₂, R₇, R₉ and R₁₄ represent a hydrogen atom (feature f2). It is also preferred that R₃, R₆, R₁₀ and R₁₃ represent a hydrogen atom (feature f3). More preferably, features f1 and f2 are met. Still more preferably, features f1, f2 and f3 are met.

In certain embodiments in accordance with the present invention, at least one of R₂, R₃, R₄, R₅, R₆, R₇, R₉, R₁₀, R₁₁, R₁₂, R₁₃ and R₁₄ is an electron-withdrawing group. The case being, the electron-withdrawing group can be as here above detailed.

Preferably, R₁ and R₈ are identical to each other (feature f4).

Invented compounds wherein
(i) R₂ and R₁₄ are identical to each other,
(ii) R₃ and R₁₃ are identical to each other,
(iii) R₄ and R₁₂ are identical to each other,
(iv) R₅ and R₁₁ are identical to each other,
(v) R₆ and R₁₀ are identical to each other, and
(vi) R₇ and R₉ are identical to each other are also preferred (feature f5).

Invented compounds wherein
(i) R₂ and R₉ are identical to each other,
(ii) R₃ and R₁₀ are identical to each other,
(iii) R₄ and R₁₁ are identical to each other,
(iv) R₅ and R₁₂ are identical to each other,
(v) R₆ and R₁₃ are identical to each other, and
(vi) R₇ and R₁₄ are identical to each other form still another class of preferred compounds (feature f6).

Still another preferred feature f7 is characterized as follows:
(i) R₂ and R₇ are identical to each other,
(ii) R₃ and R₆ are identical to each other,
(iii) R₄ and R₅ are identical to each other,
(iv) R₉ and R₁₄ are identical to each other,
(v) R₁₀ and R₁₃ are identical to each other, and
(vi) R₁₁ and R₁₂ are identical to each other.

Features f4, f5, f6 and f7 can be obviously combined with each other as long, and also with any one of features f1, f2 and f3. For example, combinations f4+f6, f1+f5+f7 and f1+f2+f3+f4 represent certain possible combinations.

More preferably, the invented compound complies with the following combination of features:
(i) R₁ and R₈ are identical to each other,
(ii) R₂, R₇, R₉ and R₁₄ are identical to each other,
(iii) R₃, R₆, R₁₀ and R₁₃ are identical to each other, and
(iv) R₄, R₅, R₁₁ and R₁₂ are identical to each other.

Still more preferably, the invented compound is a compound of formula II:

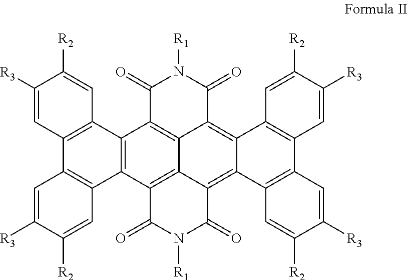

Still more preferably, the invented compound is a compound of formula III:

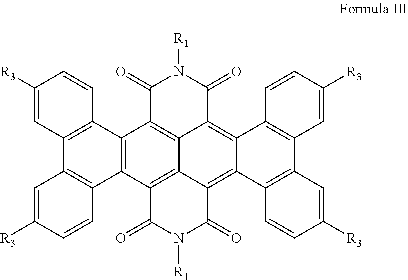

Excellent results were obtained with the compound of formula III wherein R₁ is a C₁-C₃₀ alkyl and wherein R₃ is a hydrogen atom, such as the compound of formula III wherein R₁ is n-octyl and R₃ is a hydrogen atom.

In another aspect, the present invention concerns a method for preparing at least one compound of formula I as previously defined, which comprises causing a halogenocompound of formula A

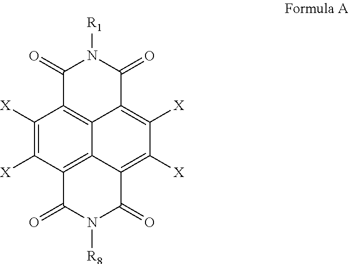

to react with a first 9-stannafluorene compound of formula B-I

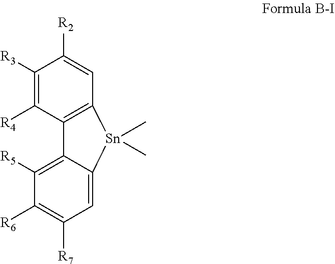

and, optionally in addition, with another 9-stannafluorene compound of formula B-II

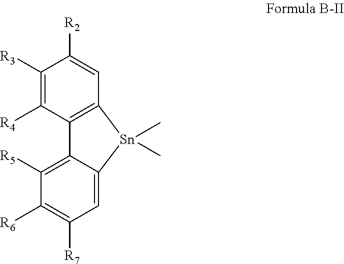

so as to form the at least one compound complying with formula I,

wherein X represents a halogen atom, preferably a bromine atom, and

R₁, R₂, R₃, R₄, R₅, R₆, R₇, R₈, R₉, R₁₀, R₁₁, R₁₂, R₁₃ and R₁₄ are also as previously defined (hereinafter, the invented method).

Preferably, the halogenocompound is of formula A-II

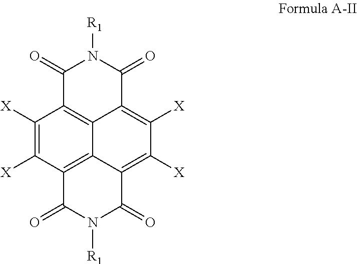

and this one is caused to react with one and only one 9-stannafluorene compound of formula B-I, to form either a mixture of two isomers of formulae IV and V

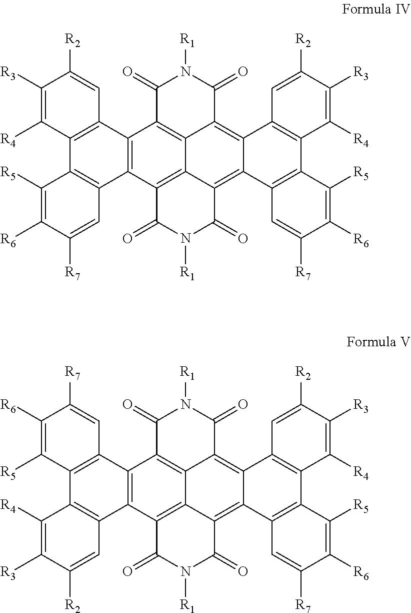

when formula B-I is asymmetric, or a single compound of formula IV=V when formula B-I is symmetric (R₂=R₇, R₃=R₆, and R₄=R₅).

In certain embodiments of the invented method, several compounds complying with formula I are formed, and at least one of them is separated from the other ones, so as to provide one and only one compound of formula I.

In certain other embodiments of the invented method, one or more compounds not complying with formula I are formed as by-products, and the at least one compound complying with formula I is separated from the by-products, so as to provide the at least one compound of formula I. The separation can be achieved by any separation technique known to the skilled person, notably by distillation or chromatography techniques such as column chromatography on silica gel.

The above two embodiments may be combined with each other.

Conditions suitable for causing the halogenocompound of formula A to react with the first 9-stannafluorene compound of formula B-I and, optionally in addition, with the other 9-stannafluorene compound of formula B-II can be easily determined and tuned by the skilled person. As commonly known by chemists, the reaction rate depends in general from the reaction temperature; the selectivity can also depend on the same parameter. The reaction of A with B-I or with B-I and B-II takes advantageously place at a temperature ranging between 0° C. and 300° C., preferably from 15° C. to 200° C., more preferably from 30° C. to 150° C., still more preferably from 40° C. to 100° C.

The reaction takes profitably place in solution. For this purpose, a suitable solvent is used. The nature of the solvent is not critical. Certain solvents in which the reaction can take place in accordance with the invented method are the previously listed suitable solvents for the invented compound. Excellent results were obtained when the halogenocompound of formula A was caused to react with the first 9-stannafluorene compound of formula B-I and, optionally in addition, with the other 9-stannafluorene compound of formula B-II in a solution comprising tetrahydrofuran.

In general, a catalyst is desirable, when not sometimes necessary. Catalysts containing palladium are preferred. [Pd (P Alk₁ (Alk₂))] catalysts, wherein Alk₁ and Alk₂, equal to or different from each other, represent a C₁-C₁₈ alkyl or a C₄-C₈ cycloalkyl group are very preferred. Alk₁ and Alk₂ are preferably equal to each other. Besides, Alk₁ and Alk₂ represent preferably a linear or branched alkyl group, more preferably a branched alkyl group. Alk₁ and Alk₂ contain preferably from 2 to 8 carbon atoms. Excellent results were obtained with [Pd (P (t-Bu)₂)] wherein t-Bu denotes a tert-butyl group.

In general, the presence of a salt of a halogen and an alkali metal proved also to be beneficial. For this salt, Cs is preferred as the alkali metal, while F is preferred as the halogen. Excellent results were obtained with CsF.

Further technical details on palladium-catalyzed double cross-coupling of 9-stannafluorene with 1,2-dihaloarenes, can be found in T. Angew. Chem., Int. Ed. 2009, 48, 7573, the whole content of which is herein incorporated by reference for all purposes. Certain technical features described therein could be advantageously applied to the presently invented method.

A third aspect of the present invention concerns a method for depositing a layer of the compound of formula I as above defined or the at least one compound of formula I prepared by the method as above defined onto a substrate, said method comprising dissolving said compound in an organic solvent (such as dichloromethane, chloroform, dichlorobenzene, toluene, tetrahydrofuran and mixtures thereof) to form a solution comprising the solvent and the compound, then applying said solution to the substrate, for example by coating or pulverizing said solution onto the substrate.

A fourth aspect of the present invention concerns the use of the compound of formula I as above defined or the use of the at least one compound of formula I prepared by the method as above defined for transporting electrons without transporting holes in a layer of an electronic device.

A fifth aspect of the present invention concerns an electronic device comprising the compound of formula I as above, defined or the at least one compound of formula I prepared by the method as above defined. The electronic device is advantageously chosen from organic electroluminescent devices, organic thermochromism elements, organic field-effect transistors and solar cells. Certain preferred electronic devices in accordance with the invention are n-channel field-effect transistors.

A last aspect of the present invention concerns the use of the compound of formula I as above defined or the use of the at least one compound of formula I prepared by the method as above defined as NIR dye, in particular in a solar cell.

The invented compound exhibits a lot of benefits. Its LUMO is advantageously below −4.00 eV. As a result thereof, the invented compound is an excellent candidate for applications wherein good air-stability is needed, in particular in electronic devices wherein electrons must be transported such as field-effect transistors. It exhibits also advantageously very good stability on exposure to light, which is also appreciated for many electronic applications, notably solar cells wherein it can be used as NIR dye. It shows also advantageous good solubility in common organic solvents such as dichloromethane, chloroform, toluene and tetrahydrofuran. The invented compound can be easily prepared via a one-pot synthesis; the invented method relies on a flexible, tunable reaction scheme which allows for the preparation of a broad set of representative compounds in accordance with the invention.

Perhaps, the most valuable and quite unexpected possible advantage of the invented compound, which by the way was not taught in the 4-page communication of the Journal of the American Chemical Society entitled “One-pot synthesis of stable NIR tetracene diimides via double cross-coupling” published prior to filing, is that its band gap is substantially higher than the band gap of the compounds of the prior art having substantially the same LUMO, which have been proposed not just for injecting and transporting electrons, but also for injecting and transporting holes, “thanks to/because of” lower band gap. A high band gap, typically of at least above 1.80 eV as profitably featured by the invented compound, is clearly a huge advantage when the compound is called to work exclusively as “n-type material”; in general, n-field-effect transistors that are prepared with the invented compound can be turned off with a clear cut from the “on” position to the “off” one.

### EXAMPLES

The examples below are extracted from the previously discussed 4-page communication of the Journal of the American Chemical Society entitled “One-pot synthesis of stable NIR tetracene diimides via double cross-coupling”.

Materials and Methods.

¹H NMR and ¹³C NMR spectra were recorded in deuterated solvents on a Bruker ADVANCE 400 NMR Spectrometer and a Bruker ADVANCE 600 NMR Spectrometer. ¹H NMR chemical shifts are reported in ppm downfield from tetramethylsilane (TMS) reference using the residual protonated solvent as an internal standard.

Mass spectra (MALDI-TOF-MS) were determined on a Bruker BIFLEX III Mass Spectrometer, and high resolution mass spectra (HRMS) were determined on IonSpec 4.7 Tesla Fourier Transform Mass Spectrometer.

Absorption spectra were measured with Hitachi (model U-30.10) UV-Vis spectrophotometer in a 1-cm quartz cell.

Cyclic voltammograms (CVs) were recorded on a Zahner IM6e electrochemical workstation using glassy carbon discs as the working electrode, Pt wire as the counter electrode, Ag/Ag⁺ electrode as the reference electrode. 0.1 M tetrabutylammonium hexafluorophosphate (TBAPF6) dissolved in CH2Cl2 (HPLC grade) was employed as the supporting electrolyte.

All chemicals were purchased from commercial suppliers and used without further purification unless otherwise specified.

N,N′-di(n-octyl)-2,3,6,7-tetrabromo-1,4,5,8-naphthalene tetracarboxylic diimide (compound 1a) was synthesized in accordance with the procedure described in D. Org. Lett. 2007, 9, 3917. N,N′-di(2,2,3,3,4,4,4-heptafluorobutyl)-2,3,6,7-tetrabromo-1,4,5,8-naphthalene tetracarboxylic diimide (compound 1 b) was synthesized in accordance with the same procedure.

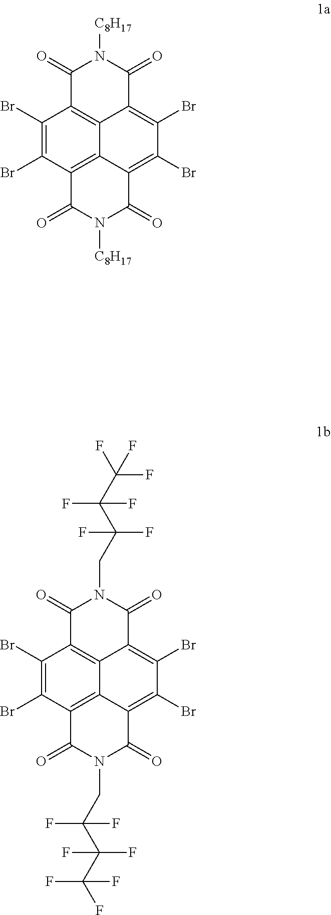

For the avoidance of doubt, in compound 1a, C₈H₁₇ is more precisely n-C₈H₁₇ (n-octyl).

Compound 11 (according to the invention) and compounds 5a, 5c, 6a and 10 (for comparison purposes), the formulae of which are shown hereunder, were prepared, characterized and tested.

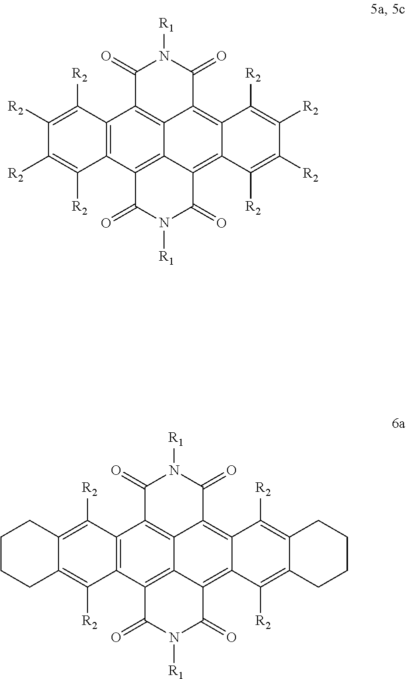

in 5a: R₁=n-C₈H₁₇ and R₂═C₂H₅ in 5a: R₁=n-C₈H₁₇ and R₂═C₂H₅ in 5c: R₁═CH₂CF₂CF₂CF₃ and R₂═C₂H₅

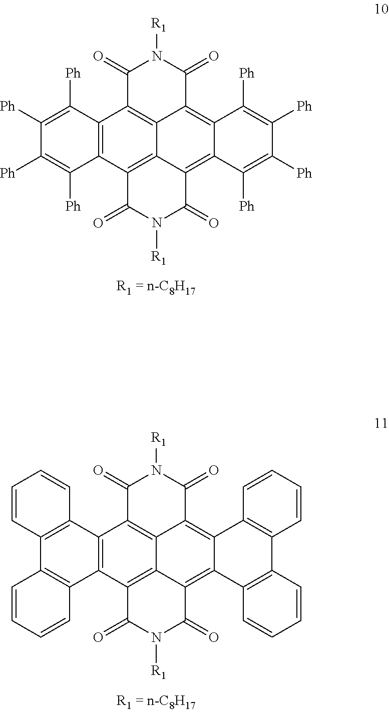

The fact that compounds 5a, 5c, 6a and 10 are provided for comparison purposes should in no way be construed as an admission from the Applicant that these compounds did form part of the prior art prior to Journal of the American Chemical Society communication entitled “One-pot synthesis of stable NIR tetracene diimides via double cross-coupling”.

Synthesis Procedure of Compounds 5a, 5c and 6a (Provided for Comparison)

To a solution of Cp₂ZrCl₂ (292 mg, 1 mmol) in 5 ml of THF was added n-BuLi (2.5M hexane solution, 0.8 ml, 2 mmol) at −78° C., and the mixture was stirred for 1 h. To the mixture was added alkyne (2 mmol) or diynes (1 mmol) and it was warmed to room temperature. After stirring for 3 h, CuCl (200 mg, 2 mmol), and 1a or 1b (0.4 mmol) were added to the mixture, which was then warmed to 50° C. for another 3 h. The mixture was quenched with aqueous HCl and extracted with dichloromethane, the combined organic phase was washed with water and dried over MgSO₄. After removal of the solvent, the residue was purified by silica gel chromatography (dichloromethane: petroether=1:2) to afford the desired compounds as a marine solid.

Synthesis Procedure of Compound 10 (for Comparison) and of Compound 11 (According to the Invention)

A mixture of 1,2,3,4-tetraphenylstannane 8 (0.51 g, 1.0 mmol) or 9-stannafluorene 9 (0.30 g, 1.0 mmol) with 2,3,6,7-tetrabromo-1,4,5,8-naphthalene tetracarboxylic imide 1a (320 mg, 0.4 mmol), Pd [P (t-Bu)₃)₂] (0.02 mol, 5 mol %), CsF (760 mg, 5 mmol) and THF (10 mL) was prepared in a glove box. The vial was taken outside the glove box and heated at 70° C. for 10 h. After cooled to room temperature, the reaction mixture was concentrated in vacuum. The crude product was purified by column chromatography on silica gel (hexane/CH₂Cl₂=1:1) to give respectively 10 (150 mg, 32%) as a carmine solid or 11 (132 mg, 42%) as a violet solid.

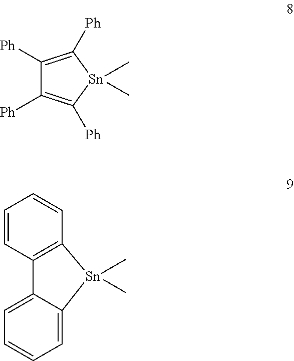 [SMILES: [CH3][Sn]1([CH3])[C](c2ccccc2)=C(c2ccccc2)C(c2ccccc2)=[C]1c1ccccc1 ; [CH3][Sn]1([CH3])[c]2ccccc2-c2cccc[c]21]

Characterization of the Compounds Using ¹H NMR, ¹³C NMR and Mass Spectrometry.

Compound 5a (176 mg, 55%): ¹H NMR (CDCl₃, 400 MHz, 298 K): δ=4.27 (m, 4H), 3.02-3.07 (m, 8H), 2.85-2.87 (m, 8H), 1.82-1.86 (m, 4H), 1.35-1.36 (m, 4H), 1.28-1.33 (m, 28H), 0.79-0.87 (s, 18H). ¹³C NMR (CDCl₃, 100 MHz, 298 K): δ=163.65, 143.40, 137.04, 136.76, 122.22, 120.64, 42.40, 31.97, 29.62, 29.36, 28.90, 27.30, 26.00, 22.79, 22.74, 16.48, 15.67, 14.21. MS (MALDI-TOF): cacld for M⁻, 814.6. found, 814.6.

Compound 5c (132 mg, 44%): ¹H NMR (CDCl3, 400 MHZ, 298 K): δ=5.04 (m, 4H), 2.87-3.01 (m, 16H), 1.26-1.34 (m, 12H), 0.81-0.84 (m, 12H).

¹³C NMR (CDCl₃, 100 MHz, 298 K): δ=162.54, 144.47, 137.38, 137.00, 122.75, 119.59, 29.74, 25.80, 22.70, 16.27, 15.46. MS (MALDI-TOF): cacld for M⁻, 954.3. found, 954.5.

Compound 6a (144 mg, 48%): ¹H NMR (CDCl₃, 400 MHZ, 298 K): δ=4.27 (m, 4H), 2.98 (m, 16H), 1.94 (m, 8H), 1.83 (m, 4H), 1.27-1.37 (m, 20H), 0.83-0.86 (m, 18H). ¹³C NMR (CDCl₃, 100 MHz, 298 K): δ=163.75, 139.71, 136.28, 136.14, 122.50, 120.35, 42.39, 31.96, 29.61, 29.36, 28.89, 27.85, 27.30, 26.65, 22.84, 22.79, 15.02, 14.22. MS (MALDI-TOF): cacld for M⁻, 810.5. found, 810.6.

Compound 10 (150 mg, 32%): ¹H NMR (CDCl₂CDCl₂, 300 MHZ, 373 K): δ=6.85-6.58 (m, 40H), 2.79 (m, 4H), 1.32-0.79 (m, 24H), 0.60 (m, 6H).

¹³C NMR (CDCl₃, 100 MHz, 298 K): δ=162.40, 142.34, 141.47, 138.74, 137.30, 136.38, 127.07, 126.36, 126.24, 124.09, 123.99, 42.37, 32.01, 29.40, 29.14, 27.40, 27.15, 22.79, 14.25. MS (MALDI-TOF): cacld for M⁻, 1198.6. found, 1198.8.

Compound 11 (132 mg, 42%): ¹H NMR (CDCl₃, 400 MHZ, 298 K): δ=8.31 (d, 2H), 8.11 (d, 2H), 7.65 (t, 4H), 7.42 (t, 4H), 4.26 (m, 4H), 1.84 (m, 4H), 1.48 (m, 4H), 1.50-1.60 (m, 4H), 1.43 (m, 16H). ¹³C NMR (CDCl₃, 100 MHz, 298 K): δ163.64, 140.04, 133.11, 132.52, 130.89, 128.70, 125.47, 125.94, 123.82, 119.55, 42.31, 31.91, 29.57, 28.61, 27.33, 22.78, 14.21. MS (MALDI-TOF): cacld for M⁻, 790.4. found, 790.4.

UV-Visible-NIR Absorption Spectra of Compounds 5a, 10 and 11.

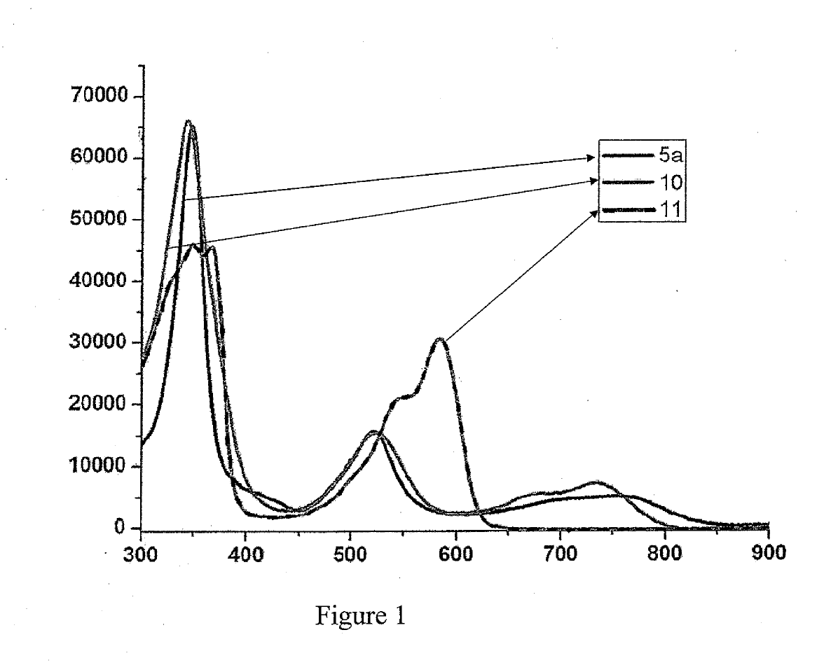

The absorption spectra of the representative tetracene tetracarboxylic diimides (TDIs) in chloroform are shown in FIG. 1, showing broad absorption which covers the whole visible and NIR region, rendering them as “full absorption dyes” and therefore potential objects in solar cells.

In FIG. 1, the X-axis is for the wavelength (expressed in nm) and the Y-axis is for the molar absorptivity ε (in L·mol⁻¹).

Compared with their all-carbon parent, tetracene (474 nm), those TDIs displayed broad and significantly red-shifted spectra and had higher molar extinction coefficient in the visible-NIR region. The remarkable difference between TDIs and tetracene in their absorption peaks and intensities reflect the substantial electronic effect of the attachment of strong electron-withdrawing diimides groups. Furthermore, in contrast with NDI (380 nm), which show absorption only in the UV region, the fusion of two additional aromatic rings also led to much more red shift in absorption as a reflection of a larger extensive conjugation over the electronic system. The absorption spectra of 11 led to a remarkable change. The purple-red solution of compound 11 exhibits a major absorption band at 584 nm with a blue shift of 176 nm relative to 10 (λₘₐₓ=740 nm), as a reflection of the interruption of tetrabenzo-units to the effective conjugation along the cores of the TDIs.

Redox Properties UV-Visible-NIR Absorption Spectra.

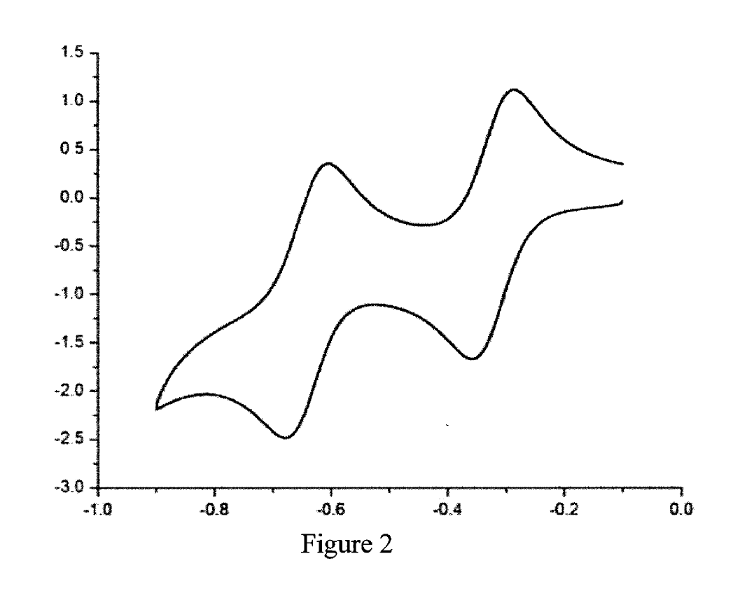

The redox properties of these TDIs were studied by cyclic voltammetry in dichloromethane (in V vs Ag/AgCl). The cyclic voltammetry curves of compound 11 (according to the invention), are provided in FIG. 2.

In FIG. 2, the X-axis is for the potential (expressed in V) and the Y-axis is for the current (expressed as 10⁻⁵ A).

The halfwave reduction potentials of the representative compounds are −0.63, −1.11 V for NDI; −0.40, −0.73 V for 5a; and −0.32, −0.64 V for 11.

The first reduction potentials of these TDIs are much less negative than those of parent NDI (see Table 1), thus, revealing the extremely strong electron-accepting abilities, and the first reduction potential of 5c is less negative by about 0.1 V than the other alkyl substituted TDIs, which is probably due to the introduction of semiperfluoroalkyl chains.

The optical band gaps and LUMO energy levels of these compounds were calculated based on UV-visible-NIR absorption data and the onset potential of the first reduction wave.

The results are made available in table 1.

TABLE 1

| | λ₁ | E₁ᵣ | LUMO | HOMO | E_{g} |
|---|---|---|---|---|---|
| | (nm)^{a} | (V)^{b} | (ev)^{c} | (eV)^{d} | (eV)^{e} |
| NDI^{f} | 380 | −0.63 | −3.90 | −7.02 | 3.12 |
| 5a | 756 | −0.40 | −4.06 | −5.54 | 1.48 |
| 6a | 780 | −0.40 | −4.06 | −5.50 | 1.44 |
| 5c | 774 | −0.29 | −4.17 | −5.50 | 1.43 |
| 10 | 737 | −0.35 | −4.12 | −5.68 | 1.56 |
| 11 | 584 | −0.32 | −4.14 | −6.13 | 1.99 |

^{a}λ₁ as peak of the visible-NIR regions.
^{b}V vs Ag/AgCl. Half wave redox potential (in V vs Ag/AgCl) measured in CH₂Cl₂ with a scan rate of 0.1 V/s.
^{c}Calculated by measuring the onset potential of the first reduction wave.
^{d}Estimated from LUMO levels and E_{g}.
^{e}Obtained from the edge of the absorption spectra.
^{f}NDI: N,N′-di(n-octyl)-naphthalene-1,2:6,7-tetracarboxylic diimide

In contrast with NDI which has a LUMO of −3.90 and exhibits poor air-stability, the LUMO of all the tested TDIs (whatever according to the invention or provided for comparison) fall well below −4.00 eV; accordingly, all the tested TDIs are deemed to exhibit high air-stability.

From a global perspective, the band gaps of all these TDIs are much smaller than the band gap of NDI (λₘₐₓ=380 nm, optical gap=3.12 eV), which could be related to the increasing conjugation length. Notably, the value of the gaps are comparable to the HOMO-LUMO gap of heptacene, which shows that the introduction of electron-withdrawing tetracarboxylic diimides can reduce the gaps more efficiently than the fusion of rings.

Comparing now more closely the band gaps of the TDIs with each other, all the compounds provided for comparison exhibit band gaps E_{g} substantially lower than 1.80 eV, in the range 1.43-1.56 eV, which are comparable to the band gap of TT-TDI (E_{g}=1.52 eV).

It will be apparent to those skilled in the art that various modifications and variations can be made to the present invention without departing from the spirit and scope of the invention. Thus, it is intended that the present disclosure covers the modifications and variations of this invention, provided they come within the scope of the appended claims and their equivalents.

1. A compound of formula I: 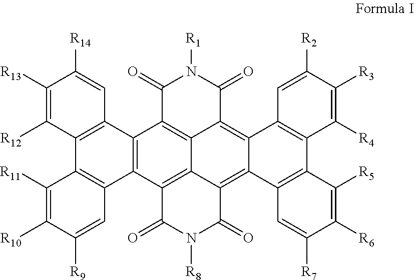
wherein:
R₁ and R₈, identical to or different from each other, are chosen from hydrogen, and C₁-C₃₀ organic groups, and
R₂, R₃, R₄, R₅, R₆, R₇, R₉, R₁₀, R₁₁, R₁₂, R₁₃ and R₁₄, identical to or different from each other and identical to or different from R₁ and R₈, are chosen from hydrogen, halogens and C₁-C₃₀ organic groups.

2. The compound according to claim 1, wherein said compound is not the compound of formula I wherein R₁ and R₈ are n-octyl and wherein R₂, R₃, R₄, R₅, R₆, R₇, R₉, R₁₀, R₁₁, R₁₂ and R₁₃ are hydrogen.

3. The compound according to claim 1, wherein the C₁-C₃₀ organic groups are electron-withdrawing groups chosen from cyano, C₁-C₃₀ acyls, halogenos, C₁-C₃₀ perhalogenocarbyls, C₁-C₃₀ perhalogeno-oxycarbyls, C₁-C₃₀ partially halogenated hydrocarbyls having a halogen atom over hydrogen atom molar ratio of at least 0.50 and C₁-C₃₀ partially halogenated oxyhydrocarbyls having a halogen atom over hydrogen atom molar ratio of at least 0.50;
or
wherein the C₁-C₃₀ organic groups are solubilizing groups chosen from C₁-C₃₀ hydrocarbyls, C₁-C₃₀ oxyhydrocarbyls, C₁-C₃₀ partially halogenated hydrocarbyls having a halogen atom over hydrogen molar ratio below 0.50 and C₁-C₃₀ partially halogenated oxyhydrocarbyls having a halogen atom over hydrogen molar ratio below 0.50.

4. (canceled)

5. The compound according to claim 1, wherein R₁ and R₈ are solubilizing groups.

6. The compound according to claim 5, wherein R₁ and R₈ are C₁-C₃₀ alkyls.

7. The compound according to claim 1, wherein R₄, R₅, R₁₁ and R₁₂ each represent a hydrogen atom.

8. The compound according to claim 7, wherein R₂, R₇, R₉ and R₁₄ each represent a hydrogen atom.

9. The compound according to claim 8, wherein R₃, R₆, R₁₀ and R₁₃ each represent a hydrogen atom.

10. The compound according to claim 1, wherein at least one of R₂, R₃, R₄, R₅, R₆, R₇, R₉, R₁₀, R₁₁, R₁₂, R₁₃ and R₁₄ is an electron-withdrawing group.

11. The compound according to claim 1, wherein R₁ and R₈ are identical to each other.

12. The compound according to claim 1, wherein
(i) R₂ and R₁₄ are identical to each other,
(ii) R₃ and R₁₃ are identical to each other,
(iii) R₄ and R₁₂ are identical to each other,
(iv) R₅ and R₁₁ are identical to each other,
(v) R₆ and R₁₀ are identical to each other, and
(vi) R₇ and R₉ are identical to each other
or
wherein
(i) R₂ and R₉ are identical to each other,
(ii) R₃ and R₁₀ are identical to each other,
(iii) R₄ and R₁₁ are identical to each other,
(iv) R₅ and R₁₂ are identical to each other,
(V) R₆ and R₁₃ are identical to each other, and
(vi) R₇ and R₁₄ are identical to each other;
or
wherein
(i) R₂ and R₇ are identical to each other,
(ii) R₃ and R₆ are identical to each other,
(iii) R₄ and R₅ are identical to each other,
(iv) R₉ and R₁₄ are identical to each other,
(v) R₁₀ and R₁₃ are identical to each other, and
(vi) R₁₁ and R₁₂ are identical to each other;
or
wherein
(i) R₁ and R₈ are identical to each other,
(ii) R₂, R₇, R₉ and R₁₄ are identical to each other,
(iii) R₃, R₆, R₁₀ and R₁₃ are identical to each other, and
(iv) R₄, R₅, R₁₁ and R₁₂ are identical to each other.

13. (canceled)

14. (canceled)

15. The compound according to claim 12, wherein,
said compound is a compound of formula II: 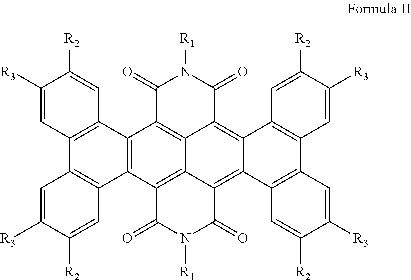
or a compound of formula III:

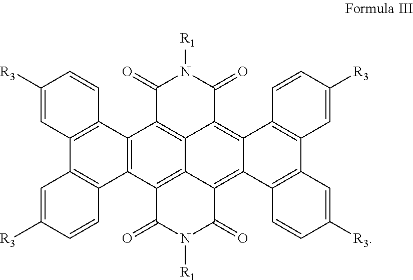

16. A method for preparing at least one compound of formula I according to claim 1, the method comprising causing a halogenocompound of formula A 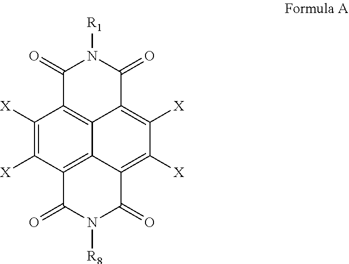
to react with a first 9-stannafluorene compound of formula B-I 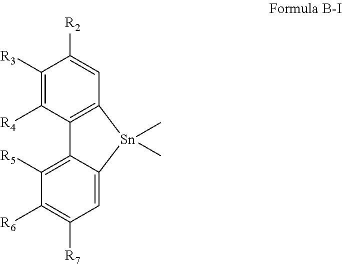
and, optionally in addition, with another 9-stannafluorene compound of formula B-II 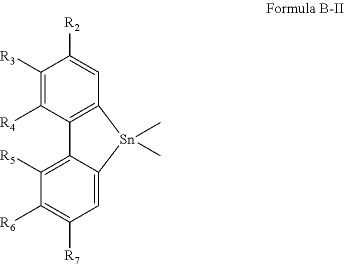
so as to form the at least one compound complying with formula I,
wherein X represents a halogen atom, and
R₁, R₂, R₃, R₄, R₅, R₆, R₇, R₈, R₉, R₁₀, R₁₁, R₁₂, R₁₃ and R₁₄ are as previously defined.

17. The method according to claim 16, wherein the halogenocompound is of formula A-II 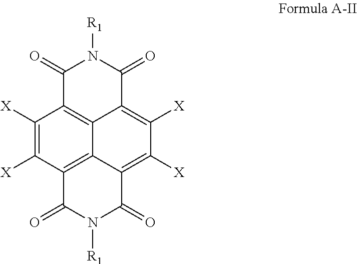
and said halogenocompound of formula A-II is caused to react with one and only one 9-stannafluorene compound of formula B-I, to form either a mixture of two isomers of formulae IV and V 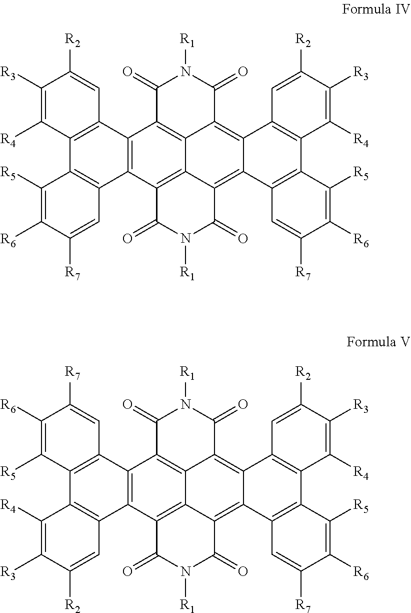
when formula B-I is asymmetrical, or a single compound of formula IV=V when formula B-I is symmetrical (R₂=R₇, R₃=R₆, and R₄=R₅).

18. The method according to claim 16, wherein several compounds complying with formula I are formed, and at least one of them is separated from the other ones, so as to provide one and only one compound of formula I.

19. The method according to claim 16, wherein one or more compounds not complying with formula I are formed as by-products, and the at least one compound complying with formula I is separated from the by-products, so as to provide the at least one compound of formula I.

20. The method according to claim 18, wherein the separation is achieved by distillation or chromatography techniques.

21. A method for depositing a layer of the compound of formula I according to claim 1 onto a substrate, said method comprising dissolving said compound in an organic solvent to form a solution comprising the solvent and the compound, then applying said solution to the substrate.

22. A layer for transporting electrons without transporting holes of an electronic device, the layer comprising the compound of formula I according to claim 1.

23. An electronic device comprising the compound of formula I according to claim 1, said electronic device being chosen from organic electroluminescent devices, organic thermochromism elements, organic field effect transistors and solar cells.

24. The electronic device according to claim 23, wherein said electronic device is a n-channel field-effect transistor.

25. A NIR dye comprising the compound of formula I according to claim 1.
